(19) United States
(12) Patent Application Publication
(10) Pub. No.: US 2004/0064107 A1
(43) Pub. Date: Apr. 1, 2004

(54) Locking design for a needle head

(76) Inventor: Pi-Chang Lo, Taoyuan City (TW)

Correspondence Address: M.K. Meditech Co., Ltd., P.O. Box No. 6-57, Chung-Ho, Taipei, TW

(21) Appl. No.: 10/255,594

(22) Filed: Sep. 27, 2002

(51) Int. Cl. A61M 5/00

(52) U.S. Cl. 604/240

(57) ABSTRACT

A locking design aims to dispose a retaining structure between a needle base and a needle head of a syringe needle barrel for eliminating any medical contamination caused by reusing the needle head. Mainly, a needle base looping disposed at the front end of the needle barrel extends convexly to form an encircling body; the inner lateral of the encircling body forms a retaining structure with a drop to prevent the jointed needle head and needle base from being pulled apart thereby allowing the needle head to be used only once but not for repetitive application through recycling.

### BACKGROUND OF THE INVENTION

1) Field of the Invention

The present invention relates to a locking design for a needle head, more particularly to a design for compulsorily allowing only a single use of the needle head; wherein the corresponding engagement between the needle head and a needle base defines a retaining structure with a drop on the looping of the needle base to make the needle head not recyclable for repetitive application after being used once so as to achieve the requirement of using the needle head only once.

2) Description of the Prior Art

The primary purpose of using a medical apparatus only once is to prevent medical contamination. Since most of the fetal diseases are infected through blood, it is obvious important to require syringes, blood drawing tools and other related apparatuses to be used only once. Therefore, many self-destructive or safety needle barrels are invented for preventing the reuse of the needle apparatuses.

However, all of the designs of the needle apparatuses are originated from the operation habits of medical workers and then sequentially select a destroyable push rod, needle barrel, needle head or rubber head for composing the syringe structure. The purpose thereof is to eliminate the possibility of reusing the related elements and the danger of medical contamination.

The needle head contacts a human body the first during the application of a needle apparatus. The structure of a needle head includes a hollow metal needle fixedly disposed at a fixed joint; the hollow metal needle deeply pierces into a blood vessel or a subcutaneous tissue for injecting the medicine or drawing the blood. Therefore, the metal needle has to be kept in an absolute clean condition for preventing any contamination. However, the hollow metal needle has a fine hollow hole which is more difficult for cleansing than the other elements of the needle apparatus. That is the reason why the needle head should not be reused.

### SUMMARY OF THE INVENTION

The primary objective of the present invention of a locking design for a needle head is to effectively fix the needle head onto a needle base such that the needle head is unable to separate from the needle base thereby eliminating the reuse of the needle head.

The present invention mainly to design a needle base looping disposed at the front end of a needle barrel for extending necessarily and convexly to form an encircling body; the inner lateral of the encircling body forms a retaining structure with a drop to disable the jointed needle head and needle base to separate thereby allowing the needle head to be used only once but not for repetitive use through recycling.

To enable a further understanding of the structural features and the technical contents of the present invention, the brief description of the drawings below is followed by the detailed description of the preferred embodiment.

### BRIEF DESCRIPTION OF THE DRAWINGS

FIG. 1 is an exploded drawing of elements of a conventional syringe.

FIG. 2A is an exploded drawing of the structures of a conventional needle base and needle head.

FIG. 2B is an exploded drawing of the structures of another conventional needle base and needle head.

FIG. 3 is a pictorial and exploded drawing of an exemplary embodiment of the present invention of a locking design for a needle head.

FIG. 4A is the first schematic drawing of the movement of the present invention of a locking design for a needle head.

FIG. 4B is the second schematic drawing of the movement of the present invention of a locking design for a needle head.

FIG. 5A is a pictorial and exploded drawing of another exemplary embodiment of the present invention of a locking design for a needle head.

FIG. 5B is an exploded and schematic drawing of another exemplary embodiment of the present invention of a locking design for a needle head.

FIG. 6A is the first schematic drawing of another assembled exemplary embodiment of the present invention of a locking design for a needle head.

FIG. 6B is the second schematic drawing of another assembled exemplary embodiment of the present invention of a locking design for a needle head.

### DETAILED DESCRIPTION OF THE PREFERRED EMBODIMENTS

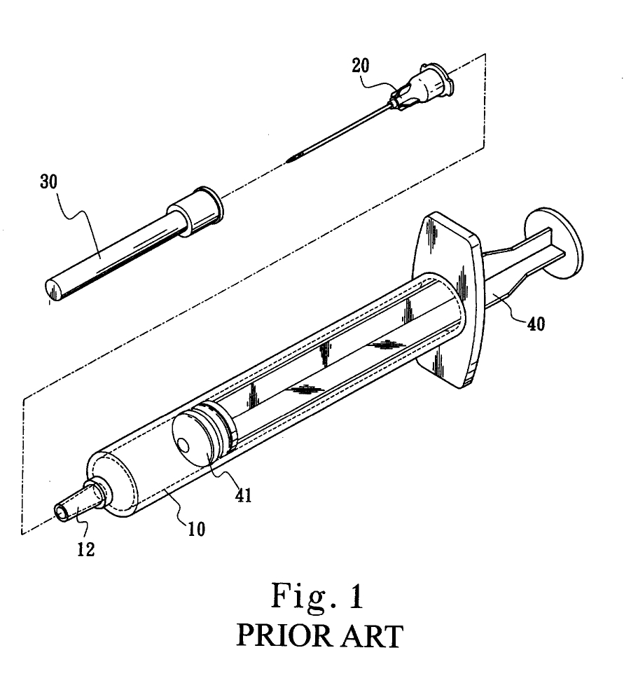

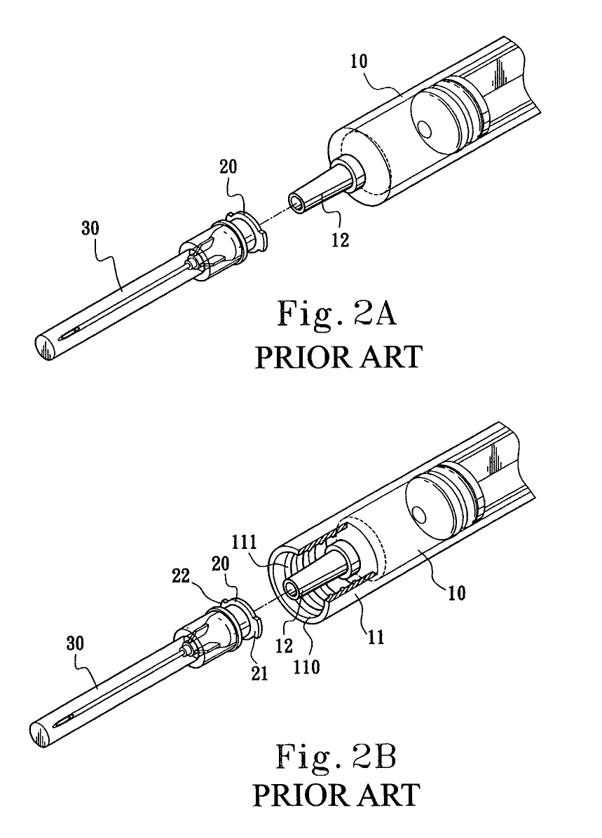

Referring to FIGS. 1, 2A and 2B, a traditional syringe mainly includes a hollow needle barrel (10), a needle head (20), a needle case (30) and a push rod (40) disposed with a rubber head (41); wherein the front end of the needle barrel (10) narrows suddenly to define a drop and form a needle base (12) to be forcefully tightened and then fixedly connected to insert the needle case (30).

However, in order to effectively engage the needle head (20) and the needle base (12) at the front end of the traditional syringe (10), another locking method is developed to fixedly rotate the needle head (20) onto the needle barrel (10). Mainly, the periphery of the needle base (12) at the front end of the needle barrel (10) extends to form an encircling body (11); the inner lateral (110) of the encircling body (11) is disposed with slant threads (11) for threading in retaining lugs (21, 2) of the needle head (20); the threading force more effectively and fixedly engages the needle head (20) and the needle base (12).

Furthermore, in order to allow the needle head (20) to be used only once, the fixedly engaged needle head (20) and needle base (12) are tightly locked and retained to disable the needle head (20) to detach from the needle base (12) thereby eliminate any repetitive use.

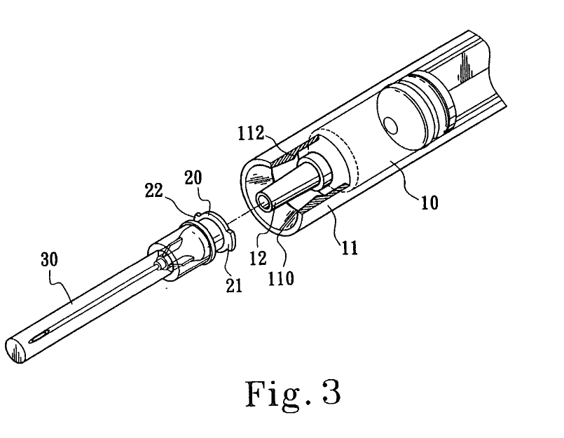

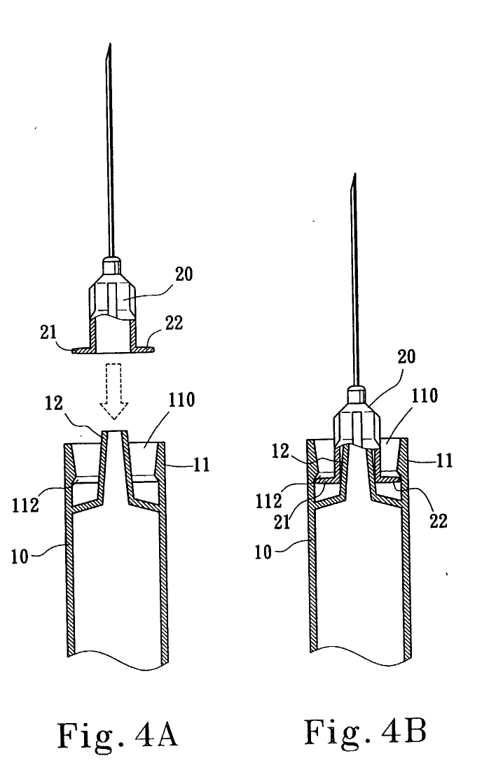

FIGS. 3, 4A and 4B show the preferred embodiments of the present invention. Mainly, the needle head (20) forcefully and tightly engages the needle base (12); wherein the front end of the needle barrel (10) necessarily extends to form the encircling body (11) which is in the concentric circles of the needle base (12); the inner lateral of the encircling body (110) gradually tapers from the end portion thereof to form a retaining ring (112); the needle head (20) is pushed toward the inner lateral (110) of the encircling body (11) when it is pushed to engage the needle base (12); at this time, the retaining lugs (21, 22) of the needle head (20) drop into the retaining ring (112) on the inner lateral (110) to form a retaining state thereby limiting the needle head (20) to tightly engage only unidirectionally but not separate toward a reverse direction. Therefore, the needle head (20) can be mounted only once but not recycled for repetitive use.

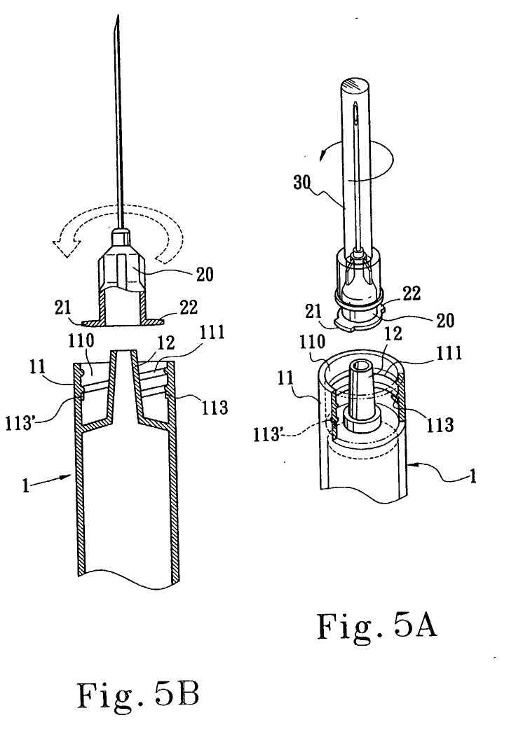

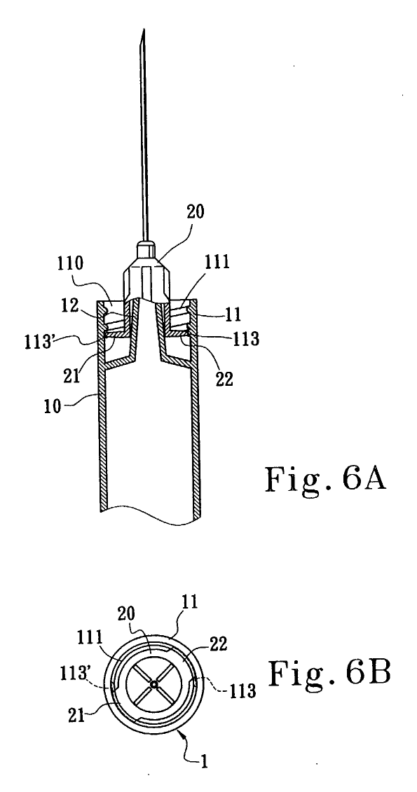

FIGS. 5A and 5B show another embodiment of the present invention. Mainly, the needle head (20) is designed to engage the needle base (12) by a rotation method; the front end of the needle barrel (10) extends to form the encircling body (11) which is in the concentric circles of the needle base (12); the looping on the inner lateral (110) thereof is disposed with slant threads (111) for threading in the retaining lugs (21, 22) of the needle head (20); the rotating force more effectively engages the needle head (20) with the needle base (12). The distal end of the slant threads (111) is disposed with corresponding retaining tabs (113, 113′) expanding to form a retaining structure capable of only sliding unidirectionally. As indicated in FIGS. 6-1 and 6-2, the encircling body (11) of the needle head (20) must thread into the slant threads (111) from an opening end thereof when the needle head (20) engages the needle base (12). However, once the retaining lugs (21, 22) of the needle head (20) thread into the retaining tabs (113, 113′), they form a locked retaining state to disable the needle head (20) to detach from the needle base (12) thereby eliminating the possibility of recycling the needle head (20) for repetitive use.

The technique and operation method of the present invention have not changed an operator's customary steps and operation procedures. Therefore, no training is required to operate the present invention for effectively preventing any dishonest manufacture from recycling the needle head (20) and causing the horrible danger.

It is of course to be understood that the embodiment described herein is merely illustrative of the principles of the invention and that a wide variety of modifications thereto may be effected by persons skilled in the art without departing from the spirit and scope of the invention as set forth in the following claims.

1. A locking design for a needle head, more particularly a design allowing needle head and a needle base of a needle apparatus to engage only once and preventing them from separation thereby eliminating any possibility of recycling the needle head for repetitive use; wherein the needle apparatus comprises a hollow needle barrel, a needle head, a needle case, a push rod and a rubber head inserted at the front end of the push rod; the present invention is characterized that a needle base looping disposed at the front end of the needle barrel extends to form an encircling body; wherein the inner lateral of the encircling body is disposed with a retaining structure with a drop to retain retaining lugs of the needle head and the retaining structure thereby preventing separation after the needle head and needle base fixedly engage so as to achieve the objective of using the needle head only once and eliminating the possibility of recycling the needle head for repetitive use.

2. The locking design for a needle head according to claim 1, wherein the looping extended from the front end of the needle barrel must belong to the concentric circles of the needle base.

3. The locking design for a needle head according to claim 1, wherein the retaining lugs of the needle head correspond partially or are of an annular structure.

4. The locking design for a needle head according to claim 1, wherein the retaining structure on the inner lateral of the encircling body tapers inwardly from the end portion of the inner lateral of the encircling body to form a slant and conical toric and retaining rings at a proper interval distance; when the needle head pushes toward the inner lateral of the encircling body, the retaining lugs of the needle head drop into the retaining rings to retain such that the needle head is limited to make only unidirectional tightening and engagement and unable to separate in a reverse direction.

5. The locking design for a needle head according to claim 1, wherein another exemplary embodiment of the retaining structure on the inner lateral of the encircling body comprises slant threads formed on the looping thereof for threading in the retaining lugs on the needle head; the distal end of the slant threads is disposed with corresponding retaining tabs expanding to form a retaining structure capable of only sliding unidirectionally such that the needle head is limited to the engagement through unidirectional rotation and unable to separate in a reverse direction.
